(19) United States
(12) Patent Application Publication
(10) Pub. No.: US 2012/0022174 A1
(43) Pub. Date: Jan. 26, 2012

(54) FISCHER-TROPSCH SYNTHESIS CATALYST, PREPARATION AND APPLICATION THEREOF

(76) Inventors: Yong Yang, Taiyuan (CN); Baoshan Wu, Taiyuan (CN); Yongwang Li, Taiyuan (CN); Hongwel Xiang, Taiyuan (CN)

(21) Appl. No.: 13/259,356

(22) PCT Filed: Apr. 8, 2010
(86) PCT No.: PCT/CN10/71629

§ 371 Date: Sep. 23, 2011

(30) Foreign Application Priority Data
Apr. 22, 2009 (CN) 200910133993.9

(51) Int. Cl. C07C 27/06; B01J 21/04; B01J 21/12

(52) U.S. Cl. 518/721; 502/241; 502/307; 502/243

(57) ABSTRACT

A micro-spherical Fe-based catalyst for a slurry bed Fischer-Tropsch synthesis (FTS) comprises Fe as its active component, a transitional metal promoter M, a structure promoter S and a K promoter. The transitional metal promoter M is one or more selected from the group consisting of Mn, Cr and Zn, and the structure promoter S is SiO₂ and/or Al₂O₃. The weight ratio of the catalyst components is Fe: transitional metal promoter: structure promoter: K=100:1-50:1-50:0.5-10. Preparation method of the catalyst comprises: adding the structure promoter S into a mixed solution of Fe/M nitrates, then co-precipitating with ammonia water to produce a slurry, filtering and washing the slurry to produce a filter cake, adding the required amount of the K promoter and water to the filter cake, pulping and spray drying, and roasting to produce the micro-spherical Fe-based catalyst for the slurry bed Fischer-Tropsch synthesis. The catalyst has good abrasion resistance and narrow particle size distribution, furthermore, it has high conversion capability of synthesis gas, good product selectivity and high space time yield, and the catalyst also can be used for the slurry bed Fischer-Tropsch synthesis in a wide temperature range.

### FIELD OF THE INVENTION

The present invention relates to a high performance catalyst for Fischer-Tropsch synthesis (F-T synthesis, FTS) in a slurry bed reactor with a wide temperature range, a preparation method and an application thereof in the slurry bed FTS reaction.

### BACKGROUND OF THE INVENTION

The FTS reaction is a process to convert carbon-containing materials (coal, natural gas and biomass, etc.) via syngas (CO+H₂) into hydrocarbons and other chemicals. The typical catalysts for converting syngas include iron, cobalt, ruthenium, nickel and other transitional metals in group VIII. Among the above mentioned catalysts, the ruthenium-based catalyst is expensive, and the methanation reaction of nickel-based catalyst tends to overact. Therefore, only Fe-based and Co-based catalysts have the potential for application in industry. The Co-based catalyst has low activity for water-gas-shift (WGS) reaction, thus it is suitable for the conversion of the natural gas derived syngas with H₂/CO molar ratio of about 2.0. The Fe-based catalyst has high activity for WGS reaction and high adaptability for the various ratio of H₂/CO of syngas. Meanwhile, Fe is low in price relative to other transitional metals in group VIII, therefore the Fe-based catalyst is widely used in the FTS. At present, the industrial Fe-based FTS catalyst is usually prepared by the methods for preparing supported catalysts, fused iron catalysts and co-precipitation catalysts etc.

Generally, the catalysts prepared by the fused-iron process are suitable for the circulating fluidized bed reactor for the FTS reaction at a temperature range of 320-350° C. The supported catalysts and co-precipitated catalysts are respectively used in the fixed bed reactor and an advanced slurry-bed reactor of the FTS reaction (220-250° C.). Compared with the fluidized bed reactor and the fixed-bed reactor, the slurry-bed reactor has the following advantages: low cost, easy operation, simplicity for catalysts displacement, good capability of heat transmission and high unit yield. In the present publications and applications, however, the key performances related to the slurry-bed Fe-based catalyst for the FTS reaction, such as activity, abrasion resistance, stability, product selectivity, space time yield, and production capacity, are low. In order to improve the parameters of these products, many domestic and foreign groups in industry and academy have conducted lots of work in research and development of the Fe-based catalysts for the slurry-bed FTS reaction. However, the advancement in the performance of the Fe-based catalyst is undesirable. In U.S. patents U.S. Pat. No. 6,265,451 and U.S. Pat. No. 6,277,895 and China patent CN1803281A, a modified Raney method was used to prepare a catalyst with C₃⁺ space time yield of only 0.26 g/g catalyst/h and an excessively high C₁₋₂ selectivity up to 9.76 wt %. U.S. patent U.S. Pat. No. 7,067,562 published a preparation method for a precipitated catalyst (100 Fe/5 Cu (1-2 Ag/Ca)/0.2-4.2 K(or 1.2-4 Li, 1-2 Ag)/10-25 SiO₂ in weight ratio) and evaluated the catalyst respectively in fixed-bed and slurry-bed reactors. However, this catalyst exhibited a highest C₅⁺ space time yield of only 0.23 g/g catalyst/h. Meanwhile, the catalyst could be only operated at a temperature range of 230-240° C. with methane selectivity also up to 4-10 wt %. Proper amount addition of the transitional metal promoter (such as Mn) can significantly improve catalytic performance of the Fe-based F-T synthesis catalyst in its activity and olefin selectivity. (Appl. Catal. A : Gen. 284 (2005) 105 and 266 (2004) 181; Catal. Today 106 (2004) 170). A kind of precipitated Fe—Mn F-T synthesis catalyst containing a promoter of Cu and K was reported in the U.S. patent U.S. Pat. No. 4,621,102 and U.S. Pat. No. 5,118,715, which exhibited a high reactivity and low CH₄ selectivity under catalytic performance test in both slurry bed reactor and fixed bed reactor with syngas of H₂/CO=2.0 as feed gas. However, without the addition of the structure promoter, the above-mentioned catalyst did not have practical feasibility because of poor abrasion resistance, while the relevant index data such as catalyst strength, space time yield and production capacity were not provided either. A kind of molecular sieve supported Fe—Mn fixed bed catalyst was introduced in U.S. patent U.S. Pat. No. 4,340,503, whereas, it had high CH₄ selectivity and low activity.

In Chinese patent CN1817451A, a series of precipitated Fe/Cu/Cr/K/Na catalysts suitable for high temperature fluidized bed F-T synthesis process was illustrated. Nevertheless, this kind of catalyst was of low activity and rather poor target product selectivity, with less than 50% syngas conversion and more than 10% CH₄ selectivity under such reaction conditions: 350° C., 1400-1450 ml/ml catalyst/h, 2.5 MPa and syngas of H₂/CO=3.0 as feeding gas.

Up to now, there are no patent reports on advanced slurry bed matched catalysts with high comprehensive performance: high activity, ideal product distribution, high abrasion resistance. Meanwhile, the slurry bed catalysts now available can only be used under low reaction temperature (220-240° C.), the operating temperature cannot be increased, which goes against the enhancement of the overall energy conversion efficiency in the F-T synthesis process.

In view of the operation characteristics of the F-T synthesis reactor as well as the deficiency of the catalyst in the prior art, the applicants find that the fully dispersive and stabilized active phase of the catalysts and the high stability of the active phase and the catalyst structure in the process of the F-T synthesis reaction can be achieved by optimally adding a certain amount of both transitional metal promoter and structure promoter (SiO₂ and/or Al₂O₃) which can form a stable structure with Fe. Thereby, a micro-spherical slurry bed F-T synthesis catalyst with wider operating temperature range (240-280° C.) is prepared, which is also characterized by high activity (high space time yield), high stability (high production capacity), ideal product selectivity as well as high abrasion resistance and strength.

### DETAILED DESCRIPTION OF THE INVENTION

The objective of the present invention is to provide a micro-spherical Fe-based catalyst suitable for the slurry bed F-T synthesis process, the catalyst comprises Fe as its active component, characterized in that the catalyst further comprises a transitional metal promoter M, a structure promoter S and a K promoter, the transitional metal promoter M is one or more selected from the group consisting of Mn, Cr and Zn, the structure promoter S is SiO₂ and/or Al₂O₃, wherein the weight ratio of Al₂O₃ to SiO₂ is not more than 0.5; the weight ratio of the components is Fe:M:S:K=100:1-50:1-50:0.5-10; wherein the metal components are calculated based on metal elements; the structure promoter is calculated based on oxides. Wherein, the amount of the transitional metal promoter M is the total amount of all the transitional metal promoters, and the amount of the structure promoter S is the sum of all the structure promoters.

Preferably, the weight ratio of each component of the micro-spherical Fe-based catalyst according to the present invention is Fe:M:S:K=100: 4-40:5-40:1-7.

In the catalyst according to the present invention, the transitional metal promoter M is preferably selected from combinations comprising two or more elements of Mn, Cr and Zn; each component can exist in any proportion when the transitional metal promoter M mentioned above comprises two or more elements.

In the catalyst according to the present invention, Al₂O₃ and SiO₂, two components of the structure promoter, can be mixed at any weight ratio; preferably the weight ratio (Al₂O₃/SiO₂) is not more than 0.5, more preferably not more than 0.3.

The other objective of the present invention is to provide the preparation method of the catalyst mentioned above. In the method, metal Fe, transitional metal M and nitric acid or the solution of the corresponding metal nitrates are used as raw materials, the routine co-precipitation method in the art is used to prepare the catalyst.

The preparation method for the catalyst above-mentioned comprises the following steps:

(1) according to the required proportion of the components, preparing a solution of metal nitrates by using metal Fe, transitional metal promoter M and nitric acid as raw materials; or preparing a mixed solution of metal nitrates by directly dissolving the metal nitrates; and adding the structure promoter S into the solution of metal nitrates; then directly precipitating the mixed solution; or precipitating the mixed solution after adding the structure promoter S;

(2) co-precipitating the solution of metal nitrates prepared in the step (1) to produce a precipitated slurry by using ammonia water as a precipitant;

(3) washing and filtering the precipitated slurry prepared in the step (2) to obtain a filter cake;

(4) adding the required amount of potassium salt as the K promoter and deionized water into the filter cake, pulping to obtain a slurry, and adjusting the pH value of the slurry to 4-10, then emulsifying the slurry to obtain a catalyst slurry;

(5) molding the catalyst slurry prepared by the step (4) by spray-drying, and roasting the molded catalyst to obtain the catalyst.

More specifically, the detailed preparation method for the catalyst according to the present invention comprises the following steps:

(1) according to the required proportion of the components, preparing a solution of metal nitrates by using metal Fe, transition metal promoter M and nitric acid as raw materials; or preparing a mixed solution of metal nitrates by directly dissolving the metal nitrates; the solution of the metal nitrates is in a total concentration of 5-45 wt % nitrates; and adding the structure promoter S into the solution of metal nitrates;

(2) co-precipitating the solution of metal nitrates prepared in the step (1) to produce a precipitated slurry by using ammonium water in a concentration of 1-25 wt % as a precipitant, the precipitation temperature is 20-95° C.; the precipitation time is 5-120 min; aging for 5-120 min after precipitation and the final pH value of the precipitated slurry is 5-10;

(3) washing and filtering the precipitated slurry prepared in the step (2) to obtain a filter cake with a solid content of 5-60 wt %;

(4) adding the required amount of potassium salt and deionized water into the filter cake, pulping to obtain a slurry, and adjusting the pH value of the slurry to 4-10, then emulsifying the slurry to obtain a catalyst slurry with a solid content of 3-50 wt %;

(5) molding the catalyst slurry prepared in the step (4) by spray-drying the catalyst slurry in a pressurized spray-drying tower, the conditions for spray-drying are as follows: an inlet air temperature of 150-450° C. and an outlet air temperature of 70-150° C.; and then roasting the molded catalyst at a temperature of 300-750° C. for 1-10 hours to obtain the desired catalyst; wherein the addition of the structure promoter S in the step (1) is changed to be performed in the step (4); or respectively adding part of the structure promoter in the steps (1) and (4). In the preparation method described above, “the required proportion” or “the required amount” refers to the description of the weight proportion between components of the catalyst in the present invention. In the process of preparation, the added amount of the raw materials and the proportion thereof are based on the principle of ensuring the ratio of each component in the final catalyst to meet the requirement described above.

In the preparation method mentioned above, the structure promoter S can also be added in the step (4) instead of step (1), that is, the structure promoter S is added into the filter cake together with deionized water and potassium salt in the step (4), followed by pulping; or respectively adding part of the structure promoter S in the step (4) and the step (1); more preferably, all of the structure promoter S is added in the step (1), or adding part of the structure promoter in the step (1) and step (4) respectively. In the case that part of the structure promoter is added in the step (1) and step (4) respectively, the weight ratio between Fe and the structure promoter in the final solution of the metal nitrates is not less than 100/30 after adding in the step (1), more preferably not less than 100/25.

In the preparation method mentioned above, the solution of the metal nitrates in the step (1) can be prepared by using metal Fe, the transitional metal M and nitric acid as raw materials, or by directly dissolving the metal nitrates, preferably, the mixed solution of the metal nitrates is prepared by directly dissolving the metal nitrates; the mixed solution of the metal nitrates prepared in the step (1) is in a total concentration of 5-45 wt %, preferably 10-40 wt %.

In the step (1) and/or step (4), silica sol and/or alumina sol which are the precursors of the structure promoter are used as raw materials in order to introduce the structure promoter SiO2 and/or Al₂O₃. To be specific, the raw material for the structure promoter SiO₂ is silica sol or potash water glass (i.e. potassium silicate) and the raw material for the structure promoter Al₂O₃ is alumina sol. The silica sol is also called silicic acid sol, which is a colloid solution of multi-molecular polymer of silicic acid. Preferably, the silica sol is acidic silica sol or alkaline silica sol; the alumina sol is hydrated alumina.

When using potash water glass as the raw material of SiO₂, the amount of metal potassium should be included in the total amount of the K promoter.

In the step (2), a continuous co-precipitation method is adapted in the precipitation process of the mixed solution of the metal nitrates and the ammonia water. The ammonia water is in a concentration of 1-25 wt %, preferably 5-20 wt %; the precipitation temperature is 20-95° C., preferably 50-90° C., and the pH value in the precipitation process is 6.0-9.5. The precipitation time is 5-120 min and ageing for 5-120 min after precipitation. The final pH value is 5-10.

In the step (3), the solid content in the filter cake obtained after washing and filtering the slurry is 5-60 wt %, preferably 15-50 wt %. The ammonium nitrate content in the filter cake is 0.1-2.5 wt %, preferably 0.01-5.0 wt %.

In the step (4), the potassium salt added as the K promoter is one selected from the group consisting of potassium bicarbonate, potassium acetate, organic sylvite and potash water glass, preferably selected from the group consisting of potassium bicarbonate, potassium acetate and potash water glass. The pH value of the slurry obtained after adding potash water glass and deionized water is 5.0-9.5; wherein the solid content in the slurry is 3-50 wt %, preferably 10-40 wt %.

When using potash water glass as the K promoter, the content of SiO₂ therein should be included in the total amount of the structure promoter.

In the step (5), the spray-drying process can be carried out in a conventional facility of the prior art, preferably in a pressurized spray-drying tower; wherein the process conditions can be those often used in the facility and method, for example, in the spray-drying process, the inlet air temperature is 150-450° C. and the outlet air temperature is 70-150° C.; preferably the inlet air temperature is 180-420° C. and the outlet air temperature is 85-130° C.; the roasting process can also be carried out in the conventional facility of the prior art, preferably in an air atmosphere; for example, the roasting temperature is 300-750° C. and the roasting time is 1-10 hours; preferably the roasting temperature is 350-700° C. and the roasting time is 2-8 hours.

Compared with the prior art, the catalyst and its preparation method in the present invention have the following advantages:
(1) By means of adding the transitional metal, the active component Fe is well stabilized and dispersed and the electronic structure on the catalyst surface is improved, thereby significantly enhancing the activity of the catalyst (conversion capability of the syngas) and optimizing the selectivity of the catalyst for the products such as hydrocarbons and by-products or the like.
(2) By way of adding the structure promoters Al₂O₃ and/or SiO₂ with a certain proportion in the process of precipitation and/or molding, the formation, structure and stability of the active component in the reduced catalyst can be adjusted and controlled effectively, thereby enhancing the structure, running stability and abrasion resistance of the catalyst, meanwhile being able to carry out the F-T synthesis process in a wider temperature range.
(3) When using the catalyst of the present invention for the F-T synthesis process, the production conditions are mild, the process is simple, the raw materials of metal and the promoters are low in price and the production cost is low.

### EXAMPLES

The technical solutions of the present invention will be described in detail according to the following examples which are used for exemplifying the present invention, but not intended to limit the protection scope of the present invention in any way. The percentage involved in the examples refers to weight percentage.

### Example 1

2000 kg of Fe(NO3)₃.9H₂O, 36 kg of Mn(NO₃)₃ aqueous solution in a concentration of 50 wt %, 10.65 kg of Cr(NO3)₃.3H₂O and 6.3 kg of Zn(NO₃)₂.6H₂O were dissolved in 1050 kg of deionized water. After fully dissolved, into the mixed solution of the metal nitrates were added 45 kg of silica sol with a SiO₂ content of 30 wt % and 1.2 kg of alumina sol with an Al₂O_{'}content of 25 wt %. The obtained mixed solution was heated to 50° C. under stirring, and the total concentration of the nitrates in the mixed solution was 12.22 wt % with the weight ratio of each component being Fe:Mn:Cr:Zn:SiO₂:Al₂O₃=100:2.0:0.5:0.5:4.88:0.11.

Meanwhile, an ammonia water solution in a concentration of 10.0 wt % was prepared and heated to 30° C. 1500 kg of deionized water was put into the precipitation pot in advance, and then preheated to 50° C., when reaching the setting temperature, ammonia water was co-precipitated with the above-mentioned mixed solution by co-flowing process. The temperature of the slurry in the precipitation pot was maintained at 50° C. and the pH value of the slurry was maintained at 6.5±0.3. The mixing and co-precipitation process was completed within 15 min, followed by standing still and aging for 60 min.

The aged slurry was washed with deionized water until the content of NH₄NO₃ in the slurry was 0.10 wt %, then filtrated to obtain a filter cake with a solid content of 48.5 wt %. Into the obtained filter cake, potassium acetate solution (which was prepared by dissolving 8.35 kg of potassium acetate in 412 kg of deionized water) was added, then sufficiently pulped to obtain a slurry, and adjusting the pH value of the slurry to 5.2, a solid content of the obtained slurry was 35.0 wt %.

The slurry prepared above was spray dried in a pressurized spray drying tower with an inlet air temperature of 180° C. and an outlet air temperature of 85° C. The dried molded catalyst was placed in the roaster, and roasted at 350° C. for 8 hours in air atmosphere to produce the required Fe-based catalyst, the weight ratio among each component in the catalyst was Fe:Mn:Cr:Zn:SiO₂:Al₂O₃:K=100:2.0:0.5:0.5:4.88:0.11:1.2. This catalyst is labeled as A.

### Example 2

2000 kg of Fe(NO₃)₃.9H₂O, 324 kg of Mn(NO₃)₃ aqueous solution in a concentration of 50 wt %, 127.5 kg of Cr(NO₃)₃.3H₂O and 201.5 kg of Zn(NO₃)₂.6H₂O were dissolved in 1360 kg of deionized water. After fully dissolved, into this mixed solution of the metals nitrates were added 24.0 kg of alumina sol with an Al₂O₃ content of 25.0 wt %. The obtained mixed solution was heated to 90° C. under stirring, and the total concentration of the nitrates in mixed solution was 39.6 wt % with the weight ratio of each component being Fe:Mn:Cr:Zn:Al₂O₃=100:20.0:5.0:15.0:2.0.

Meanwhile, an ammonia water solution in a concentration of 20.0 wt % was prepared and heated to 60° C. 1500 kg of deionized water was put into the precipitation pot in advance, and then preheated to 90° C., when reaching the setting temperature, ammonia water was co-precipitated with the above-mentioned mixed solution of the metal nitrates by co-flowing process. The temperature of the slurry in the precipitation pot was maintained at 90° C. and the pH value of the slurry was maintained at 9.0±0.3. The mixing and co-precipitation process was completed within 20 min, followed by standing still and aging for 20 min.

The aged slurry was washed with deionized water until the content of NH₄NO₃ was 2.45 wt %, then filtrated to obtain a filter cake with a solid content of 36.5 wt %. Into the obtained filter cake were added a certain amount of potassium acetate solution, alumina sol with an Al₂O₃ content of 25 wt % and deionized water, then sufficiently pulped to obtain a slurry, and adjusting the pH value of the slurry to 9.2, a solid content of the obtained slurry was 30.0 wt %. The amount of the added water glass and alkaline silica sol was based on the principle of meeting the final content ratio of each structure promoter.

The above-prepared slurry was spray dried in a pressurized spray drying tower with an inlet air temperature of 210° C. and an outlet air temperature of 95° C. The dried molded catalyst was placed in the roaster, and roasted at 700° C. for 2 hours in air atmosphere to produce the required Fe-based catalyst, the weight ratio among each component in the catalyst was Fe:Mn:Cr:Zn:Al₂O₃:K=100:20.0:5.0:15.0:20:4.5. This catalyst is labeled as B.

### Example 3

2000 kg of Fe(NO₃)₃.9H₂O, 270 kg of Mn(NO₃)₃ aqueous solution in a concentration of 50 wt %, 127.5 kg of Cr(NO₃)₃.3H₂O were dissolved in 2500 kg of deionized water. After fully dissolved, the obtained mixed solution was heated to 80° C. under stirring, and the total concentration of the nitrates in the mixed solution was 29.5 wt % with the weight ratio of each component being Fe:Mn:Cr=100:15.0:6.0.

Meanwhile, an ammonia water solution in a concentration of 12.5 wt % was prepared and heated to 40° C. 1500 kg of deionized water was put into the precipitation pot in advance, and then preheated to 80° C., when reaching the setting temperature, ammonia water was co-precipitated with the above-mentioned mixed solution of the metal nitrates by co-flowing process. The temperature of the slurry in the precipitation pot was maintained at 80° C. and the pH value of the slurry was maintained at 8.5±0.3. The mixing and co-precipitation process was completed within 30 min, followed by standing still and aging for 30 min.

The aged slurry was washed with deionized water until the content of NH₄NO₃ was 1.5 wt %, then filtrated to obtain a filter cake with a solid content of 16.5 wt %. Into the obtained filter cake were added a certain amount of KHCO₃, alkaline silica sol, alumina sol and deionized water, then sufficiently pulped to obtain a slurry, and adjusting the pH value of the slurry to 8.8, a solid content of the obtained slurry was 12.0 wt %. The amount of the added alkaline silica sol and alumina sol was based on the principle of meeting the final content ratio of each structure promoter.

The slurry above-prepared was spray dried in a pressurized spray drying tower with an inlet air temperature of 400° C. and an outlet air temperature of 105° C. The dried molded catalyst was placed in the roaster, and roasted at 600° C. for 7.5 hours in air atmosphere to produce the required Fe-based catalyst, the weight ratio among each component in the catalyst was Fe:Mn:Cr:SiO₂:Al₂O₃:K=100:15.0:6.0:20.0:6.0:6.0. This catalyst is labeled as C.

### Example 4

2000 kg of Fe(NO₃)₃.9H₂O, 216 kg of Mn(NO₃)₃ aqueous solution in a concentration of 50 wt %, 216 kg of acidic silica sol with a SiO₂ content of 25.0 wt % were dissolved in 4000 kg of deionized water. After fully dissolved, the obtained mixed solution was heated to 65° C. under stirring, and the total concentration of the nitrates in the mixed solution was 21.0 wt % with the weight ratio of each component being Fe:Mn:SiO₂=100:12.0:19.5.

Meanwhile, an ammonia water solution in a concentration of 15.0 wt % was prepared and heated to 40° C. 1500 kg of deionized water was put into the precipitation pot in advance, and then preheated to 65° C., when reaching the setting temperature, ammonia water was co-precipitated with the above-mentioned mixed solution of the metal nitrates by co-flowing process. The temperature of the slurry in the precipitation pot was maintained at 65° C. and the pH value of the slurry was maintained at 7.55±0.3. The mixing and co-precipitation process was completed within 20 min, followed by standing still and aging for 30 min.

The aged slurry was washed with deionized water until the content of NH₄NO₃ was 1.0 wt %, then filtrated to obtain a filter cake with a solid content of 26.5 wt %. Into the obtained filter cake were added a certain amount of potash water glass with a modulus of 4.0, alkaline silica sol and deionized water, then sufficiently pulped to obtain a slurry, and adjusting the pH value of the slurry to 8.2, a solid content of the obtained slurry was 25.0 wt %. The amount of the added potash water glass and alkaline silica sol was based on the principle of meeting the final content ratio of each structure promoter.

The above-prepared slurry was spray dried in a pressurized spray drying tower with an inlet air temperature of 350° C. and an outlet air temperature of 125° C. The dried molded catalyst was placed in the roaster, and roasted at 500° C. for 3.5 hours in air atmosphere to produce the required Fe-based catalyst, the weight ratio among each component in the catalyst was Fe:Mn:SiO₂:K=100:12.0:38.5:6.8. This catalyst is labeled as D.

### Example 5

2000 kg of Fe(NO₃)₃.9H₂O, 126.0 kg of Mn(NO₃)₃ aqueous solution in a concentration of 50 wt %, 37.8 kg of Zn(NO₃)₂.6H₂O were dissolved in 2000 kg of deionized water. After fully dissolved, into this mixed solution of the metal nitrates were added 33.2 kg of alumina sol with an Al₂O₃ content of 25.0 wt %. The obtained mixed solution was heated to 80° C. under stirring, and the total concentration of the nitrates in the mixed solution was 30.9 wt % with the weight ratio of each component being Fe:Mn:Zn:Al₂O₃=100:7.0:3.0:3.0.

Meanwhile, an ammonia water solution with a concentration of 13.5 wt % was prepared and heated to 45° C. 1500 kg of deionized water was put into the precipitation pot in advance, and then preheated to 80° C., when reaching the setting temperature, ammonia water was co-precipitated with the above-mentioned mixed solution of the metal nitrates by co-flowing process to obtain a slurry. The temperature of the slurry in the precipitation pot was maintained at 80° C. and the pH value of the slurry was maintained at 7.5±0.3. The mixing and co-precipitation process was completed within 25 min, followed by standing still and aging for 15 min.

The aged slurry was washed with deionized water until the content of NH₄NO₃ was 0.5 wt %, then filtrated to obtain a filter cake with a solid content of 38.5 wt %. Into the obtained filter cake were added a certain amount of potassium carbonate, acidic silica sol, alumina sol and deionized water, then sufficiently pulped to obtain a slurry, adjusting the pH value of the slurry to 7.2, a solid content of the obtained slurry was 32.0 wt %. The amount of the added acidic silica sol and alumina sol was based on the principle of meeting the final content ratio of each structure promoter.

The above-prepared slurry was spray dried in a pressurized spray drying tower with an inlet air temperature of 250° C. and an outlet air temperature of 100° C. The dried molded catalyst was placed in the roaster, and roasted at 550° C. for 6 hours in air atmosphere to produce the required Fe-based catalyst, the weight ratio among each component in the catalyst was Fe:Mn:Zn:SiO₂:Al₂O₃:K=100:7.0:3.0:4.0:6.0:3.5. This catalyst is labeled as E.

### Example 6

2000 kg of Fe(NO₃)₃.9H₂O, 170.0 kg Cr(NO₃)₃.3H₂O and 75.6 kg of Zn(NO₃)₂.6H₂O were dissolved in 1700 kg of deionized water. After fully dissolved, into this mixed solution of the metal nitrates were added 18.5 kg of silica sol with a SiO₂ content of 30 wt % and 16.6 kg of alumina sol with an Al₂O₃ content of 25 wt %, the obtained mixed solution was heated to 60° C. under stirring, and the total concentration of the nitrates in the mixed solution was 35.3 wt % with the weight ratio of each component being Fe:Cr:Zn:SiO₂:Al₂O₃=100:8.0:6.0:2.0:1.5.

Meanwhile, an ammonia water solution with a concentration of 18.0 wt % was prepared and heated to 40° C. 1500 kg of deionized water was put into the precipitation pot in advance, and then preheated to 60° C., when reaching the setting temperature, ammonia water was co-precipitated with the above-mentioned mixed solution of the metal nitrates by co-flowing process. The temperature of the slurry in the precipitation pot was maintained at 60° C. and the pH value of the slurry was maintained at 7.0±0.3. The mixing and co-precipitation process was completed within 22 min, followed by standing still and aging for 35 min.

The aged slurry was washed with deionized water until the content of NH₄NO₃ was 1.2 wt %, then filtrated to obtain a filter cake with a solid content of 25.5 wt %. Into the obtained filter cake were added a certain amount of potash water glass with a modulus of 3.3, alumina sol, acidic silica sol and deionized water, then sufficiently pulped to obtain a slurry, adjusting the pH value to 8.5, a solid content of the obtained slurry was 18.0 wt %. The amount of the added potash water glass, acidic silica sol and alumina sol was based on the principle of meeting the final content ratio of each structure promoter.

The above-prepared slurry was spray dried in a pressurized spray drying tower with an inlet air temperature of 320° C. and an outlet air temperature of 120° C. The dried molded catalyst was placed in the roaster, and roasted at 600° C. for 6 hours in air atmosphere to produce the required Fe-based catalyst, the weight ratio among each component in the catalyst was Fe:Cr:Zn:SiO₂:Al₂O₃:K=100:8.0:6.0:10.5:4.5:4.0. This catalyst is labeled as F.

### Example 7

300 kg of iron, 300 kg of manganese, 18 kg of chromium and 27 kg of zinc were reacted with a proper amount of HNO₃ solution in a concentration of 50 wt %. The tail gas was adsorbed with deionized water by pressurized sprinkle to produce nitric acid for repeated use. The total concentration of the nitrates in the mixed solution prepared above was 32.2 wt %. Into this mixed solution of the metal nitrates were added 35.0 kg of silica sol with a SiO₂ content of 30 wt % and 24.0 kg of alumina sol with an Al₂O₃ content of 25 wt %. The obtained mixed solution was heated to 85° C. under stirring, and the weight ratio of each components in the obtained mixed solution of the metal nitrates was Fe:Mn:Cr:Zn:SiO₂:Al₂O₃=100:18.0:6.0:9.0:3.5:2.0.

Meanwhile, an ammonia water solution in a concentration of 16.0 wt % was prepared and heated to 60° C. 1500 kg of deionized water was put into the precipitation pot in advance, and then preheated to 85° C., when reaching the setting temperature, ammonia water was co-precipitated with the above-mentioned mixed solution of the metal nitrates by co-flowing process. The temperature of the slurry in the precipitation pot was maintained at 85° C. and the pH value of the slurry was maintained at 7.0±0.3. The mixing and co-precipitation process was completed within 27 min, followed by standing still and aging for 90 min. The aged slurry was washed with deionized water until the content of NH₄NO₃ was 1.5 wt %, then filtrated to obtain a filter cake with a solid content of 35.0 wt %. Into the obtained filter cake were added a certain amount of potash water glass with a modulus of 3.3, alumina sol, acidic silica sol and deionized water, then sufficiently pulped to obtain a slurry, adjusting the pH value of the slurry to 8.0, a solid content of the obtained slurry was 28.0 wt %. The amount of the added potash water glass, acidic silica sol and alumina sol was based on the principle of meeting the final content ratio of each structure promoter.

The above-prepared slurry was spray dried in a pressurized spray drying tower with an inlet air temperature of 240° C. and an outlet air temperature of 110° C. The dried molded catalyst was placed in the roaster, and roasted at 550° C. for 5 hours in air atmosphere to produce the required Fe-based catalyst, the weight ratio among each component in the catalyst was Fe:Mn:Cr:Zn:SiO₂:Al₂O₃:K=100:18.0:6.0:9.0:18.0:3.0:5.0. This catalyst is labeled as G.

The following Table 1 lists the composition and physical properties of the prepared catalysts described in the Examples 1-7.

TABLE 1
The composition and physical properties of catalysts described in the Examples 1-7

| | | Catalyst labels | | | | | | |
|---|---|---|---|---|---|---|---|---|
| Preparation conditions | | A | B | C | D | E | F | G |
| Catalyst | Fe | 100 | 100 | 100 | 100 | 100 | 100 | 100 |
| composition | Mn | 2.0 | 20.0 | 15.0 | 12.0 | 7.0 | — | 18.0 |
| (weight | Cr | 0.5 | 5.0 | 6.0 | — | — | 8.0 | 6.0 |
| ratio) | Zn | 0.5 | 15.0 | — | — | 3.0 | 6.0 | 9.0 |
| | K | 1.2 | 4.5 | 6.0 | 6.8 | 3.5 | 4.0 | 5.0 |
| | SiO₂ added before | 4.88 | — | — | 19.5 | — | 2.0 | 3.5 |
| | precipitation | | | | | | | |
| | Al₂O₃ added before | 0.11 | 2.0 | — | — | 3.0 | 1.5 | 2.0 |
| | precipitation | | | | | | | |
| | SiO₂ added before | — | — | 20.0 | 19.0 | 4.0 | 8.5 | 14.5 |
| | molding | | | | | | | |
| | Al₂O₃ added before | — | 18.0 | 6.0 | — | 3.0 | 3.0 | 1.0 |
| | molding | | | | | | | |
| | Total amount of structure | 4.99 | 20.0 | 26.0 | 38.5 | 10.0 | 15.0 | 21.0 |
| | promoters | | | | | | | |
| Preparing | Concentration of the nitrates | 12.2 | 39.6 | 29.5 | 21.0 | 30.9 | 35.3 | 32.2 |
| conditions | (wt %) | | | | | | | |
| | Concentration of ammonia | 10.0 | 20.0 | 12.5 | 15.0 | 13.5 | 18.0 | 16.0 |
| | water (wt %) | | | | | | | |
| | Temperature of the nitrates | 50 | 90 | 80 | 65 | 80 | 60 | 85 |
| | (° C.) | | | | | | | |
| | Temperature of ammonia | 30 | 60 | 40 | 40 | 45 | 40 | 60 |
| | water (° C.) | | | | | | | |
| | Synthetic temperature (° C.) | 50 | 90 | 80 | 65 | 80 | 60 | 85 |
| | Synthetic pH value | 6.5 ± 0.3 | 9.0 ± 0.3 | 8.5 ± 0.3 | 7.5 ± 0.3 | 7.5 ± 0.3 | 7.0 ± 0.3 | 9.0 ± 0.3 |
| | Synthetic time (min) | 15 | 20 | 30 | 45 | 25 | 22 | 27 |
| | Aging time (min) | 60 | 20 | 30 | 50 | 15 | 35 | 90 |
| molding and | Potassium source | Potassium | Potassium | KHCO₃ | 4.0K | K₂CO₃ | 3.3K | 3.3K |
| roasting | | acetate | acetate | | water | | water | water |
| | | | | | glass | | glass | glass |
| | SiO₂ source | Acidic | — | alkaline | alkaline | Acidic | Acidic | Acidic |
| | | silica | | silica | silica | silica | silica | silica |
| | | sol | | sol | sol | sol | sol | sol |
| | pH value of slurry | 5.2 | 9.2 | 8.8 | 8.2 | 7.2 | 8.5 | 8.0 |
| | Solid content of slurry (wt %) | 35.0 | 30.0 | 12.0 | 25.0 | 32.0 | 18.0 | 28.0 |
| | Inlet air temperature (° C.) | 180 | 210 | 400 | 350 | 250 | 320 | 240 |
| | Outlet air temperature (° C.) | 85 | 95 | 105 | 125 | 100 | 120 | 110 |
| | Roasting temperature (° C.) | 350 | 700 | 600 | 500 | 550 | 600 | 550 |
| | Roasting time (h) | 8 | 2 | 7.5 | 3.5 | 6 | 6 | 5 |
| structural | BET specific surface area | 154 | 178 | 216 | 273 | 121 | 177 | 265 |
| properties | (m²/g) | | | | | | | |
| and particle | Pore volume (cm³/g) | 0.34 | 0.35 | 0.43 | 0.52 | 0.25 | 0.38 | 0.50 |
| size | Average pore diameter (nm) | 9.21 | 9.03 | 7.45 | 6.72 | 10.25 | 8.34 | 6.50 |
| | Percentage of 30-180 μm | 95 | 94 | 89 | 96 | 97 | 93 | 94 |

### Example 8

Using the prepared catalysts described in the Examples 1-7, the F-T synthesis reaction was conducted in the slurry bed reactor under the following catalyst reduction and F-T synthesis reaction conditions. The reactivity parameters of the FT reaction are listed in Table 2.

### Catalyst Reduction Conditions:

Reducing for 5-48 hours by using syngas as a reducing gas at a temperature of 220-300° C., a pressure of 0.1-4.0 MPa, and the space velocity of 500-10000 h⁻¹.

### F-T Synthesis Reaction Conditions in Slurry Bed Reactor:

The FT reaction was conducted in a slurry bed reactor with H₂/CO ratio of 0.7-3.0 at a temperature of 240-280° C. and at a pressure of 1.0-5.0 MPa. The space velocity of the fresh air was 5000-12000 h⁻¹ and the tail gas cycle ratio was 0.5-4.0.

The data in Table 2 demonstrate a high F-T synthesis reactivity of the catalysts according to the present invention in a slurry bed reactor even at a high space velocity. The H₂ and CO conversion are both above 80% and the target hydrocarbons selectivity (C₂⁼˜C₄⁼+C₅⁺) maintains more than 90.0 wt % while CH₄ selectivity is less than 5 wt %. Particularly, C₅⁺ selectivity and yield are both very high, which is higher than 0.80 g/g catalyst/h. Therefore, the catalysts in the present invention are especially suitable for producing products such as gasoline, diesel and wax from syngas in the slurry bed reactor.

TABLE 2
Evaluation results of the catalysts in the invention

| F-T synthesis | Catalyst labels | | | | | | |
|---|---|---|---|---|---|---|---|
| reactivity | A | B | C | D | E | F | G |
| CO conversion (%) | 82.1 | 87.5 | 90.3 | 89.2 | 92.1 | 88.6 | 87.9 |
| H₂ conversion (%) | 80.4 | 85.2 | 87.6 | 88.0 | 89.3 | 85.1 | 84.9 |

Hydrocarbons selectivity (wt %)

| | | | | | | | |
|---|---|---|---|---|---|---|---|
| CH₄ | 5.0 | 3.8 | 2.2 | 4.5 | 2.8 | 1.8 | 2.0 |
| C₂~C₄ | 11.2 | 8.8 | 6.6 | 8.9 | 6.5 | 5.4 | 6.8 |
| C₅⁺ | 83.8 | 87.4 | 91.2 | 86.6 | 90.7 | 92.8 | 91.2 |
| C₂⁼~C₄⁼ + C₅⁺ | 89.9 | 92.8 | 95.5 | 91.4 | 94.8 | 96.7 | 96.0 |
| C₂⁼~C₄⁼/C₂~C₄ (%) | 54.2 | 61.2 | 65.7 | 54.1 | 62.7 | 72.5 | 71.0 |
| CO₂ selectivity | 22.5 | 20.9 | 15.8 | 13.4 | 18.5 | 19.7 | 15.8 |
| (mol %) | | | | | | | |
| Yield (C₅⁺ g/g | 0.77 | 0.87 | 1.00 | 0.96 | 0.98 | 0.95 | 0.97 |
| catalyst/h) | | | | | | | |

The embodiments have been described above in detail, and it is obvious to the person skilled in the art that many modifications and improvements can be made without departing from the basic spirit of the present invention. All of the modifications and improvements fall into the protection scope of the invention.

1. A micro-spherical Fe-based catalyst for a slurry bed Fischer-Tropsch synthesis, comprising Fe as its active component, characterized in that the catalyst further comprises a transitional metal promoter M, a structure promoter S and a K promoter, the transitional metal promoter M is one or more selected from the group consisting of Mn, Cr and Zn, the structure promoter S is SiO₂ and/or Al₂O₃; the weight ratio of the components is Fe:M:S:K=100:1-50:1-50:0.5-10; wherein the metal components are calculated based on metal elements; the structure promoter is calculated based on oxides; the weight ratio of Al₂O₃ to SiO₂ in the structure promoter S (Al₂O₃/SiO₂) is not more than 0.5.

2. The micro-spherical Fe-based catalyst according to claim 1, characterized in that the weight ratio of the components in the catalyst is Fe:M:S:K=100:4-40:5-40:1-7; and/or when the transitional metal promoter M comprises two or more kinds of metals, these metals exist in any proportion.

3. The micro-spherical Fe-based catalyst according to claim 2, characterized in that the transitional metal promoter M is a combination of two or more kinds of metals selected from the group consisting of Mn, Cr and Zn.

4. The micro-spherical Fe-based catalyst according to claim 2, characterized in that the weight ratio of Al₂O₃ to SiO₂ in the structure promoter S is not more than 0.3.

5. The micro-spherical Fe-based catalyst according to any one of claims 1-4, characterized in that the composition of the catalyst is as follows:
Fe:Mn:Cr:Zn:SiO₂:Al₂O₃:K=100:2.0:0.5:0.5:4.88:0.11:1.2;
Fe:Mn:Cr:Zn:Al₂O₃:K=100:20.0:5.0:15.0:20:4.5;
Fe:Mn:Cr:SiO₂:Al₂O₃:K=100:15.0:6.0:20.0:6.0:6.0;
Fe:Mn:SiO₂:K=100:12.0:38.5:6.8;
Fe:Mn:Zn:SiO₂:Al₂O₃:K=100:7.0:3.0:4.0:6.0:3.5;
Fe:Cr:Zn:SiO₂:Al₂O₃:K=100:8.0:6.0:10.5:4.5:4.0; or
Fe:Mn:Cr:Zn:SiO₂:Al₂O₃:K=100:18.0:6.0:9.0:18.0:3.0:5.0.

6. A method for preparing the catalyst according to any one of claims 1-5, comprising the following steps:
(1) according to the required proportion of the components, preparing a solution of metal nitrates by using metal Fe, transitional metal promoter M and nitric acid as raw materials; or preparing a mixed solution of metal nitrates by directly dissolving the metal nitrates; and adding the structure promoter S into the solution of metal nitrates;
(2) co-precipitating the solution of metal nitrates prepared in the step (1) to produce a precipitated slurry by using ammonia water as a precipitant;
(3) washing and filtering the precipitated slurry prepared in the step (2) to obtain a filter cake;
(4) adding the required amount of potassium salt as the K promoter and deionized water into the filter cake, pulping to obtain a slurry, and adjusting the pH value of the slurry to 4-10, then emulsifying the slurry to obtain a catalyst slurry;
(5) molding the catalyst slurry prepared by the step (4) by spray-drying, and roasting the molded catalyst to obtain the catalyst.

7. The method according to claim 6, the method comprises the following steps:
(1) according to the required proportion of the components, preparing a solution of metal nitrates by using metal Fe, the transition metal promoter M and nitric acid as raw materials; or preparing a mixed solution of metal nitrates by directly dissolving the metal nitrates; the solution of the metal nitrates is in a total concentration of 5-45 wt %; and adding the structure promoter S into the solution of metal nitrates;
(2) co-precipitating the solution of metal nitrates prepared in the step (1) to produce a precipitated slurry by using ammonium water in a concentration of 1-25 wt % as a precipitant, the precipitation temperature is 20-95° C.; during the co-precipitation, the pH value is kept at 6.0-9.5; aging the precipitated slurry after precipitation, the final pH value of the precipitated slurry is 5-10;
(3) washing and filtering the precipitated slurry prepared in the step (2) to obtain a filter cake with a solid content of 5-60 wt %;
(4) adding the required amount of potassium salt as the K promoter and deionized water into the filter cake, pulping to obtain a slurry, and adjusting the pH value of the slurry to 4-10, then emulsifying the slurry to obtain a catalyst slurry with a solid content of 3-50 wt %;
(5) molding the catalyst slurry prepared in the step (4) by spray-drying the catalyst slurry in a pressurized spray-drying tower, and then roasting the molded catalyst to obtain the catalyst;
wherein the addition of the structure promoter in the step (1) is changed to be performed in the step (4); or respectively adding part of the structure promoter in the steps (1) and (4).

8. The method according to claim 7, wherein in the case that the structure promoter is added by way of respectively adding part of the structure promoter in the steps (1) and (4), the weight ratio of Fe to the structure promoter in the solution of metal nitrates is not less than 100/25 after the addition of the structure promoter in the step (1).

9. The method according to any one of claims 6-8, wherein the raw material of the structure promoter SiO₂ is silica sol or potash water glass, and/or the raw material of the structure promoter Al₂O₃ is alumina sol.

10. The method according to any one of claims 6-8, characterized in that the mixed solution of metal nitrates in the step (1) is prepared by metal nitrates; preferably the mixed solution of metal nitrates is in a concentration of 10-40 wt %.

11. The method according to any one of claims 6-8, characterized in that, in the step (2), the precipitant of ammonia water is in the concentration of 5-20 wt %, and/or the precipitation temperature is 50-90° C.

12. The method according to claim 11, characterized in that, in the step (3), the filter cake obtained by washing and filtering the precipitated slurry contains less than 2.5 wt % ammonium nitrate, and/or the solid content in the filter cake is 15-50 wt %.

13. The method according to claim 12, characterized in that, the potassium salt in the step (4) is one selected from the group consisting of potassium bicarbonate, potassium acetate, organic sylvite and potash water glass; and/or the pH value of the slurry in the step (4) is 5.0-9.5; the solid content in the catalyst slurry is 10-40 wt %.

14. A use of the Fe-based catalyst according to any one of claims 1-5 in the Fischer-Tropsch synthesis reaction, preferably the Fischer-Tropsch synthesis reaction is the one which is carried out in a slurry bed at a temperature range of 240-280° C.
